(19) United States
(12) Patent Application Publication
(10) Pub. No.: US 2018/0146915 A1
(43) Pub. Date: May 31, 2018

(54) SLEEP STAGE DETERMINATION APPARATUS AND SLEEP STAGE DETERMINATION METHOD

(71) Applicant: SLEEP SYSTEM LABORATORY INC., Sarugakucho, Chiyoda-ku, Tokyo (JP)

(72) Inventor: Shin Nemoto, Sarugakucho, Chiyoda-ku, Tokyo (JP)

(21) Appl. No.: 15/567,590

(22) PCT Filed: Apr. 24, 2015
(86) PCT No.: PCT/JP2015/062513

§ 371 Date: Oct. 18, 2017

(30) Foreign Application Priority Data
Apr. 20, 2015 (JP) 2015-086307

(51) Int. Cl. A61B 5/00; A61B 5/0245; A61B 5/11; A61B 5/16

(52) U.S. Cl. CPC A61B 5/4812; A61B 5/16; A61B 5/11; A61B 5/0245

(57) ABSTRACT

A sleep stage determination apparatus is provided with: a first normalization unit that performs a first normalization processing on the intensity of a user's heartbeat signal calculated using a gain value of gain control that is performed on the heartbeat signal so that the peak value is controlled to be constant; a second normalization unit that performs a second normalization processing on the intensity of the heartbeat signal; a third normalization unit that performs a third normalization processing on a first normalized heartbeat intensity; and a sleep stage determination unit that determines the stage of sleep on the basis of the second normalized heartbeat intensity obtained by the second normalization unit and variances indicating data variations in a predetermined period calculated for the normalized heartbeat intensity data obtained by the first normalization unit and the third normalization unit.

### TECHNICAL FIELD

The present invention relates to a sleep stage determination apparatus and a sleep stage determination method for determining a sleep stage from a heartbeat signal which has been detected from heartbeat signal detection means.

### BACKGROUND ART

Conventionally, it has been known that sleep is a barometer of health, and in daily life, a person often experiences that a comfortable sleep and the subsequent good awaking makes him or her feel a sense of refreshing and realize a good health. On the other hand, when a person suffer from insomnia or have a tendency of insomnia or when a person are forced to sleep while the lifecycles at noon and night have been reversed due to working at midnight or the like, he or she often feels bad after the awaking. That is, irrespective of whether it may be conscious or unconscious, the state of sleep influences the feeling or activities at the time of the subsequent awaking, and in turn, determines the quality of activities subsequent to the awaking.

Thus, sleep is an element which has an important influence on physical activities and mental activities of a human, and if a person can sleep well, physically and mentally healthy daily activities may be guaranteed. It is known that, as long as a person sleep comfortably, he or she is in a mentally stable state, and as long as a person is in a mentally stable state, he or she sleeps comfortably. Therefore, at the time of survey of a health state of individuals, sleep is often an index for determination of the health state, and it is well known that sleep and health is closely associated with each other. Health and depth of sleep and its quality are closely associated with the feeling or mental state of the day after, and when a mental stress is felt or a physical condition is bad, a change occurs in depth of sleep or transition pattern of sleep stage, and a comfortable sleep cannot be obtained.

In a healthy sleep, a REM sleep stage and a non-REM sleep stage repeatedly appear at predetermined intervals after falling asleep; and however, when a physical condition is poor or when a mental stress is felt, it is known that the rhythm of lifecycles is distorted. Therefore, by monitoring the sleep stages during sleep at night and the pattern of generation thereof, it is possible to know a mental stress or a bad physical condition of a user.

In particular, aged persons are likely to suffer from a bad condition of sleep such as a shallow sleep and entail a problem in quality of sleep. In order to know the quality of sleep, it is possible to find out any appropriate measure or countermeasure to improve the quality of sleep by knowing transition of a sleep stage.

Conventionally, as a method for knowing a stage of sleep, there is generally known a method employing a sleep polysomnograph (PSG) which is an internationally accepted criterion for determining the depth of sleep. In the method employing PSG, the activities in the cranial nervous system at the time of sleep are estimated from brain waves, surface muscular electric potential, eye movement or the like, and a large amount of information pertinent to sleep can be thereby obtained.

However, in the method employing the PSG, measurement is performed while a lot of electrodes are attached to the user's face or body; and therefore, a heavy sense of discomfort is imparted to the user, it is difficult to obtain a natural sleep and further there is a problem that a long period of time is required to attach the electrodes, which is very cumbersome. In addition, in the method employing the PSG, it is impossible to employ data of a first day which has been measured under a different environment from that for normal sleep as a first night effect; and moreover, there is a problem that a certain period of time from a couple of days to one week is required for the user to be familiar with such an environment. Thus, a body-related and physical load imparted to the user are very great; and therefore, it is difficult to continuously use this method daily, and only the measurement over several days is permissible at best. Further, this measurement needs to be implemented by specialists who are familiar with treatment in a specific facility such as a hospital, and the equipment available for use in measurement is expensive and thus the required costs become high. Hence, it is not practical for the user to routinely use the PSG in a hospital or at home and moreover it is difficult to use the PSG for daily health care; and therefore, the PSG entails a contradiction that the PSG may be an effective therapy for sleep disorder, whereas application of the PSG to such a patient or the like per se is difficult.

Accordingly, a method for readily keeping track of a sleep stage without employing PSG is proposed. For example, there is known a method for determining a sleep stage by employing an electrocardiograph with the use of a vibration intensity measuring instrument or the like of a wristwatch type or of such a bed-laying type. However, although there is a need to keep track of three sleep stages, at least a awaking/REM sleep stage, a shallow non-REM sleep stage, and a deep non-REM sleep stage for the purpose of health care of the user, in this method, it is possible to keep track of only two stages, awaking and sleep stages.

On the other hand, in Patent Literature 1, there is disclosed a sleep stage determination method employing a window function of a predetermined time to thereby calculate a trend curve which is representative of an increment or decrement tendency of a time change in time series of physiological information such as a heart rate or the number of pulses and then determine a sleep stage on the basis of the trend curve. This method is capable of determining four sleep stages, an awaking stage, a REM sleep stage, a shallow non-REM sleep stage, and a deep non-REM sleep stage and thus can be employed for health care.

In addition, the Inventor of the present application discloses a technique of keeping track of a sleep stage in Patent Literature 2 to Patent Literature 5 or the like as well. Specifically, in Patent Literature 2, there is disclosed a technique of performing gain control so as to include a detected physiological signal in a predetermined range, calculating a signal intensity which is inversely proportional to the gain value, and employing, as an index value, a signal intensity variance which is indicative of variation of the signal intensity or a value which is derived from the signal intensity variance to determine a sleep stage. In addition, in Patent Literature 3, there is disclosed a technique of calculating a heartbeat intensity signal from a detected heartbeat signal, calculating a variance of data in a predetermined period of time of the calculated heartbeat intensity signal, and then, from a value of the variance, keeping track of activities of sympathetic nerves. Further, in Patent Literature 4, there is disclosed a technique of employing at least either one of a parameter obtained by performing fast Fourier transform of a signal at R-R intervals of a detected heartbeat signal and variation of a signal intensity which has been calculated from the heartbeat signal to thereby obtain a 6-wave component rate by frequency analysis of brain waves and employing the 6-wave component rate of the thus obtained brain waves to thereby determine a sleep stage as well. Furthermore, in Patent Literature 5, there is disclosed a technique of determining a sleep stage from a maximum value of power spectrum density which is obtained by performing fast Fourier transform of a signal at R-R intervals of a heartbeat signal.

Further, physiological signals such as heartbeat signals are various in amplitude (intensity) depending on the users or the measuring instruments, and a difference due to individuals and a difference due to the measuring instruments may arise. Therefore, in order to improve the accuracy in determination of a sleep stage by performing universal measurement, there is a need to achieve generalization while eliminating an individual difference or an instrumental difference as to a calculated intensity of a heartbeat signal, and it is known that normalization is desirable for that purpose (for example, refer to Patent Literature 6 or the like).

### CITATION LIST

### Patent Literature

Patent Literature 1: JP2001-61820A

Patent Literature 2: JP4483862B

Patent Literature 3: JP2008-73478A

Patent Literature 4: JP2009-297474A

Patent Literature 5: JP3831918B

Patent Literature 6: JP2012-65853A

### SUMMARY OF THE INVENTION

### Problems to be Solved by the Invention

As our society becomes more complicated and highly sophisticated, increasingly more persons suffer from insomnia due to a stress exerted by making an attempt to cope with that situation. At present, it is estimated that there exist about 20 to 30% of the Japanese people who suffer from insomnia and have a significant tendency of insomnia, and there increases a business situation susceptible to stress such as shiftwork due to 24-hour work with a change in social structure and under the influence of an increased competition in business activities, and it is considered that physical and mental disorders pertinent to sleep will increase more significantly.

Under such a background, there is a desire for a technique which is capable of performing measurement in a nonrestrictive manner in conformity with an international criterion for determining the depth of sleep by adopting the PSG. The sleep stages by adopting the PSG are divided into the awaking stage, the REM sleep stage, the shallow non-REM sleep stage, and the deep non-REM sleep stage as described above on the basis of brain waves, eye movement, muscle electrocardiograph or the like, and the shallow non-REM sleep stage and the deep non-REM sleep stage each are further divided into two stages. In a stable sleep of a healthy adult, it is known as an index that the awaking stage appears at a rate of 1% to 3%, the first non-REM sleep stage appears at a rate of several %, the second non-REM sleep stage appears at a rate of 50%, the third and fourth non-REM sleep stage appear at a rate of 20% to 30%, and the REM sleep stage appears at a rate of 20% to 30%, and it is known as an index that the REM sleep appears at a cycle of about 90 minutes.

In the conventional techniques included in Patent Literature 1 to Patent Literature 5 described above, there has been set forth nothing about determination of a sleep stage in conformity with the international criterion for determining the depth of sleep including a non-REM sleep stage consisting of such a plurality of stages.

In addition, as described above, in order to determine a sleep stage by universal measurement, there is a need to appropriately normalize an intensity of a physiological signal such as a heartbeat signal; and however, in the conventional techniques included in Patent Literature 6 or the like, a specific technique for performing normalization has nowhere been disclosed.

The present invention has been made in view of the circumstance described above, and it is an object of the present invention to provide a sleep stage determination apparatus and a sleep stage determination method which eliminate any physical and mental load for a user and which are inexpensive and can be routinely used by the user and further which are capable of employing an appropriate normalization technique to thereby determine a sleep stage with its high accuracy in conformity with the international criterion for determining the depth of sleep.

### Means for Solving the Problem

In order to achieve the above object, a sleep stage determination apparatus according to the present invention, for determining a sleep stage of a user on a basis of a heartbeat signal which has been detected in a noninvasive and nonrestrictive manner at a time of sleep, the sleep stage determination apparatus comprising: heartbeat signal detection means for detecting the heartbeat signal of the user in a noninvasive and nonrestrictive manner; first normalization means for performing gain control relative to the heartbeat signal that has been detected by the heartbeat signal detection means to thereby uniformly control a peak value and then applying a first normalization process to an intensity of a heartbeat signal which has been calculated by employing a value of a gain at the time; second normalization means for applying a second normalization process to the intensity of the heartbeat signal; third normalization means for applying a third normalization process to the first normalized heartbeat intensity that has been obtained by the first normalization means; variance calculation means for calculating a variance which is indicative of variation of data of a predetermined period of time as to data of the first normalized heartbeat intensity and the third normalized heartbeat intensity that have been obtained by the first normalization means and the third normalization means, respectively; and sleep stage determination means for determining a sleep stage of the user on a basis of the second normalized heartbeat intensity that has been obtained by the second normalization means and a variance of the first normalized heartbeat intensity that has been calculated by the variance calculation means and a variance of the third normalized heartbeat intensity, wherein the sleep stage determination means performs determination of an awaking stage on the basis of the second normalized heartbeat intensity that has been obtained by the second normalization means and the variance of the third normalization intensity that has been calculated by the variance calculation means, performs determination of a REM sleep stage on the basis of the second normalized heartbeat intensity that has been obtained by the second normalization means and the variance of the third normalized heartbeat intensity that has been calculated by the variance calculation means, performs determination of a deep non-REM sleep stage on the basis of the variance of the first normalized heartbeat intensity that has been calculated by the variance calculation means and the second normalized heartbeat intensity that has been obtained by the second normalization means, and determines that a remaining time interval obtained by subtracting data relative to a time interval which has been determined to be a awaking stage, data relative to a time interval which has been determined to be a REM-sleep stage, and data relative to a time interval which has been determined to be a deep-non REM sleep stage, from data relative to all of sleep time intervals is a time interval of a shallow non-REM sleep stage.

Further, in order to achieve the above object, a sleep stage determination method of determining a sleep stage of a user on the basis of a heartbeat signal which has been detected in a noninvasive and nonrestrictive manner at a time of sleep, the sleep stage determination method comprising: a heartbeat signal detection step of detecting the heartbeat signal of the user in a noninvasive and nonrestrictive manner by predetermined heartbeat signal detection means; a first normalization step of causing a processor, which performs signal processing, to perform gain control relative to the heartbeat signal that has been detected by the heartbeat signal detection step to thereby uniformly control a peak value, and then, apply a first normalization process to an intensity of a heartbeat signal which has been calculated by employing a value of a gain at the time; a second normalization step of causing the processor to apply a second normalization process to the intensity of the heartbeat signal; a third normalization step of causing the processor to apply a third normalization process to the first normalized heartbeat intensity that has been obtained in the first normalization step; a variance calculation step of causing the processor to calculate a variance which is indicative of variation of data of a predetermined period of time as to the first normalized heartbeat intensity and the third heartbeat intensity that have been obtained in the first normalization step and the third normalization step, respectively; a sleep stage determination step of causing the processor to determine a sleep stage of the user on the basis of the second normalized heartbeat intensity that has been obtained in the second normalization step and the variance of the first normalized heartbeat intensity and the variance of the third normalized heartbeat intensity that have been calculated in the variance calculation step, wherein, in the sleep stage determination step, the processor performs determination of a sleep stage on the basis of the second normalized heartbeat intensity that has been obtained in the second normalization step and the variance of the third normalized heartbeat intensity that has been calculated in the variance calculation step, performs determination of a REM sleep stage on the basis of the second normalized heartbeat intensity that has been obtained in the second normalization step and the variance of the third normalized heartbeat intensity that has been calculated in the variance calculation step, performs determination of a deep non-REM sleep stage on the basis of the variance of the first normalized heartbeat intensity that has been calculated in the variance calculation step and the second normalized heartbeat intensity that has been obtained in the second normalization step, and determines that a remaining time interval obtained by subtracting data relative to a time interval which has been determined to be a awaking stage, data relative to a time interval which has been determined to be a REM sleep stage, and data relative to a time interval which has been determined to be a deep non-REM sleep stage, from data of all of time intervals of sleep is a time interval of a shallow non-REM sleep stage.

The sleep stage determination apparatus and sleep stage determination method, according to the present invention, perform three different types of normalization processes as to data of an intensity of a heartbeat signal which has been detected in a noninvasive and nonrestrictive manner to thereby determine a sleep stage.

### Effect of the Invention

In the present invention, the sleep stage determination apparatus and method detect a heartbeat signal in a noninvasive and nonrestrictive manner and thus eliminate any physical and mental load for a user; and are inexpensive and can be routinely used by the user; and further, are capable of employing an appropriate normalization technique to thereby eliminate an individual difference or an equipment difference, and it is possible to determine a sleep stage with its high accuracy in conformity with the international criterion for determining the depth of sleep.

### BRIEF DESCRIPTION OF THE DRAWINGS

FIG. 1 is a view showing a configuration of a sleep stage determination apparatus shown as an embodiment of the present invention.

FIG. 2 is a view showing a configuration of the sleep stage determination apparatus shown as the embodiment of the present invention, and is a partially sectional view when the apparatus is seen in the direction indicated by the arrow in FIG. 1.

FIG. 3 is a view showing a time series waveform of heartbeat intensity.

FIG. 4 is a view showing a time series waveform of a first normalized heartbeat intensity.

FIG. 5 is a view showing a time series waveform of a second normalized heartbeat intensity.

FIG. 6 is a view showing a time series waveform of a third normalized heartbeat intensity.

FIG. 7 is a view of a time series wavelength of variance of the first normalized heartbeat intensity.

FIG. 8 is a view of a time series wavelength of variance of the third normalized heartbeat intensity.

FIG. 9 is a view showing a time series waveform of heartbeat intensity, and is a view for illustrating determination of an awaking stage.

FIG. 10 is a view showing a time series waveform of the third normalized heartbeat intensity, and is a view for illustrating determination of the awaking stage.

FIG. 11 is a view showing a time series waveform of variance of the third normalized heartbeat intensity, and is a view for illustrating determination of the awaking stage.

FIG. 12 is a view of a time series waveform of the second normalized heartbeat intensity, and is a view for illustrating determination of the awaking stage.

FIG. 13 is a view showing a result of determination of each sleep stage which has been obtained by a technique according to the present invention, and is a view for illustrating determination of the awaking stage.

FIG. 14 is a view showing a result of determination by adopting the PSG that has been used for comparison with FIG. 13.

FIG. 15 is a view showing a time series waveform of variance of signal intensity.

FIG. 16 is a view showing a time series waveform of LF value that corresponds to that in FIG. 15.

FIG. 17 is a view showing a time series waveform of variance of the third normalized heartbeat intensity, and is a view for illustrating determination of a REM sleep stage.

FIG. 18 is a view showing a time series value of long term movement average value of the variance shown in FIG. 17.

FIG. 19 is a view showing a time series waveform of the second normalized heartbeat intensity, and is a view for illustrating determination of a REM sleep stage.

FIG. 20 is a view showing a result of determination by the SPG that has been used for comparison with FIG. 18.

FIG. 21 is a view showing a time series waveform of a 6-wave component by analysis of brain waves.

FIG. 22 is a view showing a time series waveform of variance of the first normalized heartbeat intensity, and is a view for illustrating determination of a deep non-REM sleep stage.

FIG. 23 is a view for illustrating a secondary determination process in determination of the deep non-REM sleep stage.

FIG. 24 is a view showing a time series waveform of the second normalized heartbeat intensity, and is a view for illustrating determination of the deep non-REM sleep stage.

FIG. 25 is a view showing a result of determination of each sleep stage which has been obtained by the technique according to the present invention.

FIG. 26 is a view showing a configuration of another physiological signal detection unit.

### MODE FOR CARRYING OUT THE INVENTION

Hereinafter, a specific embodiment in which the present invention has been applied will be described in detail with reference to the drawings.

The embodiment is directed to a sleep stage determination apparatus for determining a sleep stage. In particular, the sleep stage determination apparatus enables determination of a sleep stage with a high accuracy in conformity with the international criterion for determining the depth of sleep by adopting sleep polysomnograph (PSG).

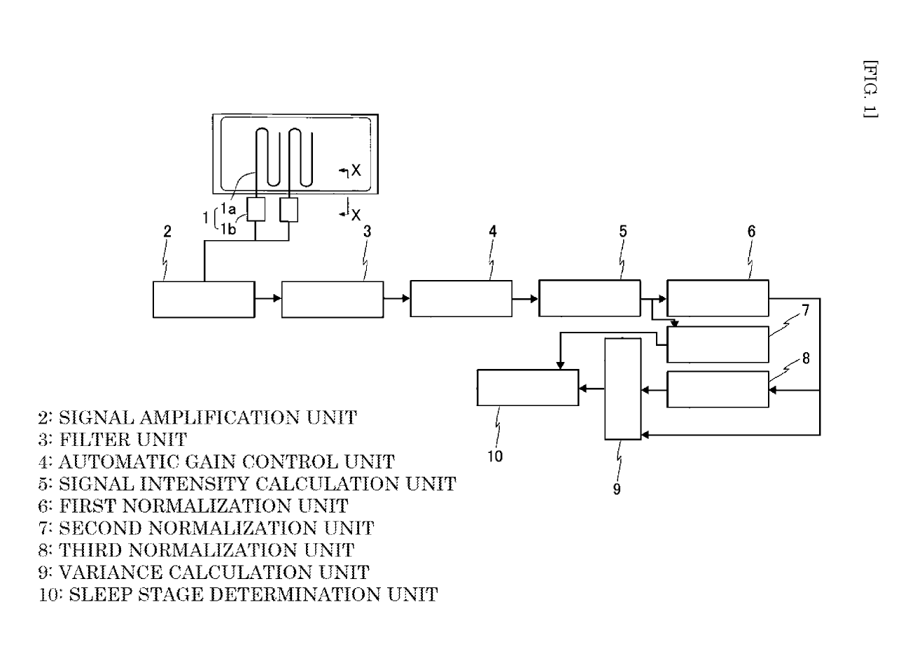

FIG. 1 shows a configuration representing a block diagram of processing operations of the sleep stage determination apparatus that is shown as an embodiment of the present invention, and FIG. 2 shows a partially sectional view when the apparatus is seen in the direction indicated by the arrow in FIG. 1. That is, the sleep stage determination apparatus is provided with: a physiological signal detection unit 1 to detect a physiological signal of a user who lays down on a bed 21; a signal amplification unit 2 to amplify the physiological signal that has been detected by the physiological signal detection unit 1; a filter unit 3 to apply a filtering process to the physiological signal that has been amplified by the signal amplification unit 2; an automatic gain control unit 4 to automatically perform gain control relative to the physiological signal that has passed through the filter unit 3; a signal intensity calculation unit 5 to calculate an intensity HI of a heartbeat signal; a first normalization unit 6 to apply a first normalization process to the heartbeat intensity HI that has been calculated by the signal intensity calculation unit 5; a second normalization unit 7 to apply a second normalization process to the heartbeat intensity HI; a third normalization unit 8 to apply a third normalization process to the first normalized heartbeat intensity HIN1 that has been obtained by the first normalization unit 6; a variance calculation unit 9 to calculate variances HIND1 and HIND3 of the first normalized heartbeat intensity HIN1 and the third normalized heartbeat intensity HIN3 that have been obtained by the first normalization unit 6 and the third normalization unit 8, respectively; and a sleep stage determination unit 10 to determine a sleep stage of a user on the basis of the second normalized variance HIN2 that has been obtained by the second normalization unit 7 and the variances HIND1 and HIND3 of the heartbeat intensities that have been calculated by the variance calculation unit 9. Incidentally, among these constituent elements, at least the signal intensity calculation unit 5, the first normalization unit 6, the second normalization unit 7, the third normalization unit 8, the variance calculation unit 9, and the sleep stage determination unit 10 each can be implemented as a program which can be executed by employing hardware such as CPU (Central Processing Unit) or memory in a computer which performs signal processing, for example, or alternatively, can be implemented by employing a dedicated processor such as a DSP (Digital Processing Unit) that has been mounted on an extension board that can be attached to the computer.

The physiological signal detection unit 1 is a sensor to detect a fine physical signal of a user in a noninvasive and nonrestrictive manner. Specifically, the physiological signal detection unit 1 is composed of: a pressure detection tube 1a; and a fine differential pressure sensor 1b which is a sensor to detect a fine pressure variation of the air that is contained in the pressure detection tube 1a, and constitutes noninvasive and nonrestrictive means for detection of a physiological signal.

As the pressure detection tube 1a, there is used the one having an appropriate resilience so that an internal pressure varies in response to a pressure variation range of a physiological signal. In addition, as the pressure detection tube 1a, there is a need to appropriately select the capacity of a hollow part of the tube in order to transmit a pressure change to a fine differential pressure sensor 1b at an appropriate speed of response. When the pressure detection tube 1a cannot meet the appropriate resilience and the capacity of the hollow part at the same time, a core wire of its appropriate thickness is provided in the hollow part of the pressure detection tube 1a all over the length, and the capacity of the hollow part can be thereby appropriately obtained.

Such a pressure detection tube 1a is disposed on a hard sheet 22 that has been provided on the bed 21. In the sleep stage determination apparatus, a cushion sheet 23 having its resilience is laid on the hard sheet 22 of which thickness is of the order of 5 mm, allowing the user to be kept at the side laying position on the pressure detection tube 1a. Incidentally, the pressure detection tube 1a may be structured so as to stabilize a position of the pressure detection tube 1a by providing a configuration to be assembled with the cushion sheet 23 or the like.

The fine differential pressure sensor 1b is a sensor to detect a fine pressure variation. In the embodiment, as the fine differential pressure sensor 1b, there is used the one of a capacitor microphone type for low frequency; and however, it is sufficient that the sensor has its appropriate resolution and dynamic range without being limitative thereto. The capacitor microphone for low frequency, which has been used in the embodiment, remarkably improves characteristics of a low frequency region by providing a chamber at a rear side of a pressure reception surface in place of the fact that a general audio microphone does not take consideration as to the low frequency region, and it is preferable to detect a fine pressure variation in the pressure detection tube 1a. In addition, this capacitor microphone is excellent in measuring a fine differential pressure; has a resolution of 0.2 Pa and a dynamic range of about 50 Pa; and has a superior performance by several times in comparison with a fine differential pressure sensor utilizing ceramics which is generally used; and it is preferable to detect a fine pressure that has been applied to the pressure detection tube 1a while a physiological signal passes through a body surface. In addition, the frequency characteristics indicate a substantially flat output value between 0.1 Hz to 30 Hz, and it is suitable to detect a fine physiological signal such as a heartbeat and breath.

In the embodiment, two sets of pressure detection tubes 1a are provided so that one of them detects a physiological signal of the user chest part and the other one detects the user buttock part, and are configured to detect a physiological signal irrespective of whatsoever the user laying posture may be. Incidentally, the sleep stage determination apparatus is configured so that the pressure detection tube 1a is disposed only at either the chest part or the buttocks part. The physiological signal that has been thus detected by the physiological signal detection unit 1 is supplied to the signal amplification unit 2. The sleep stage determination apparatus is configured to detect a physiological signal in such a noninvasive and nonrestrictive manner; can be thereby used in daily life; and is very preferable for use in an aged person, in particular.

The signal amplification unit 2 amplifies a signal which has been detected by the physiological signal detection unit 1 so as to be processed in the subsequent processing steps, and further, performs an appropriate signal reshaping process by eliminating a signal of a clearly abnormal level or the like. The physiological signal that has been amplified by the signal amplification unit 2 is supplied to the filter unit 3.

The filter unit 3 eliminates an unnecessary signal from the physiological signal that has been amplified by the signal amplification unit 2 by way of a band-pass filter or the like to thereby extract a heartbeat signal. That is, the physiological signal that has been detected by the physiological signal detection unit 1 is a signal obtained by entry of a variety of vibrations generated from a human body, and among them, in addition to a heartbeat signal, a variety of signals such as a body movement signal exerted by tossing and turning or the like is included as well. Among them, the heartbeat signal is generated as a vibration of a pressure change (that is, a blood pressure) on the basis of a heart pumping function, and is included in the physiological signals. In the sleep stage determination apparatus, this signal is extracted by the filter unit 3, and is recognized as a heartbeat signal. The heartbeat signal that has passed through the filter unit 3 is supplied to the automatic gain control unit 4. Incidentally, a sample cycle of the heartbeat signal is 4 millimeters per second.

The automatic gain control unit 4 is a so called ACG circuit to automatically perform gain control so that an output of the filter unit 3 is included in a predetermined range of a signal level. The gain control exerted by the automatic gain control unit 4 sets a gain so that the amplitude of an output signal decreases when a peak value of a signal has exceeded a predetermined upper threshold value, and sets a gain so that the amplitude increases when the peak value is lower than a predetermined lower threshold value. The automatic gain control unit 4 supplies, to the signal intensity calculation unit 5, a value (coefficient) of the gain obtained when such gain control has been performed.

The signal intensity calculation unit 5 calculates an intensity of a heartbeat signal on the basis of a coefficient of gain control that has been applied to the heartbeat signal in the automatic gain control unit 4. A value of the gain to be obtained from the automatic gain control unit 4 described above is small when the signal magnitude is large, and is set when the signal magnitude is small and thus the signal intensity is represented on the basis of a relationship which is inversely proportional to the gain value. The signal intensity calculation unit 5 supplies data of the heartbeat intensity HI to the first normalization unit 6 and the second normalization unit 7 in order to eliminate and generalize an individual difference or an equipment difference as to the calculated data of the heartbeat intensity HI.

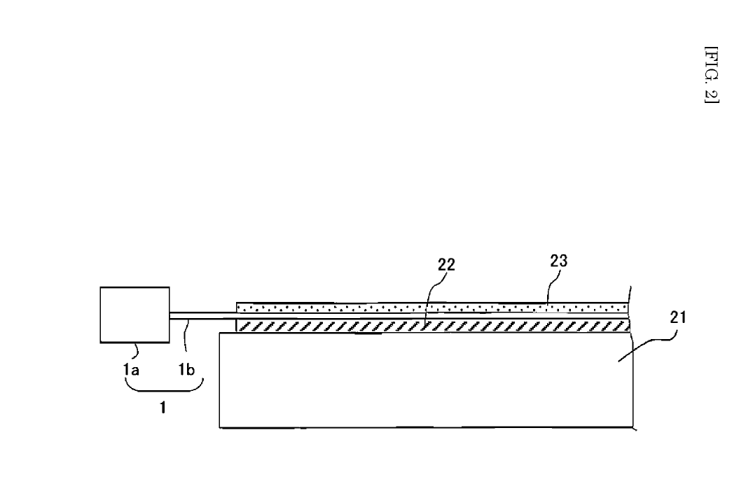

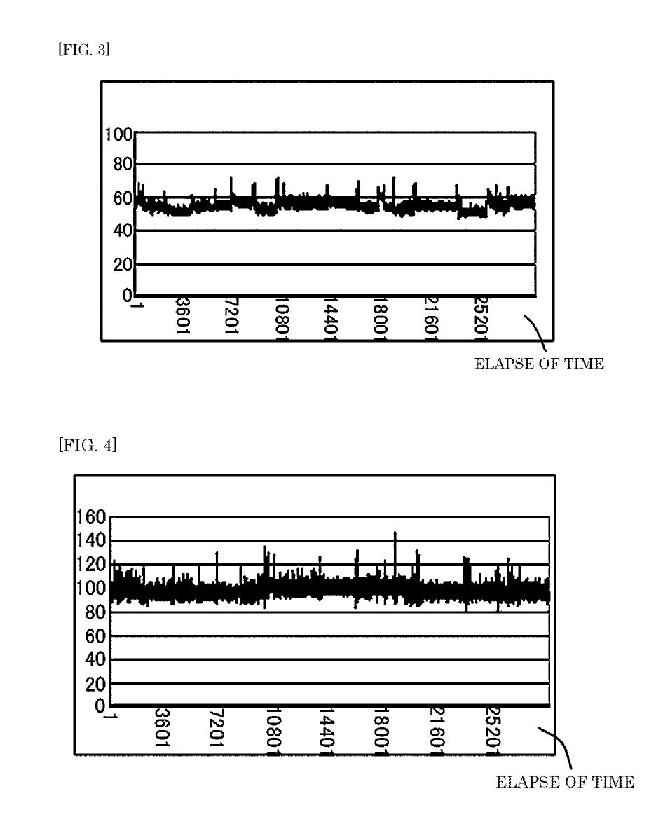

The first normalization unit 6 normalizes the data of the heartbeat intensity HI that has been calculated by the signal intensity calculation unit 5 so that the amplitude is included in a predetermined measurement range. Specifically, the first normalization unit 6 obtains a movement average value relative to the closest time intervals of 150 seconds as to the data of the heartbeat intensity HI that has been detected by the signal intensity calculation unit 5 as shown in FIG. 3, for example, and then, multiplexes, by 100 times, a value obtained by dividing the data of the heartbeat intensity HI by the movement average value to thereby perform normalization, and further, obtains the data of the first normalized heartbeat intensity HIN1 as shown in FIG. 4. The first normalization unit 6 performs such processing operation while shifting the data on a one by one second basis. Incidentally, the first normalized heartbeat intensity HIN1 is employed for determination of the deep non-REM sleep stage. The first normalization unit 6 supplies the normalized data of the first normalized heartbeat intensity HIN1 to the third normalization unit 8 and the variance calculation unit 9.

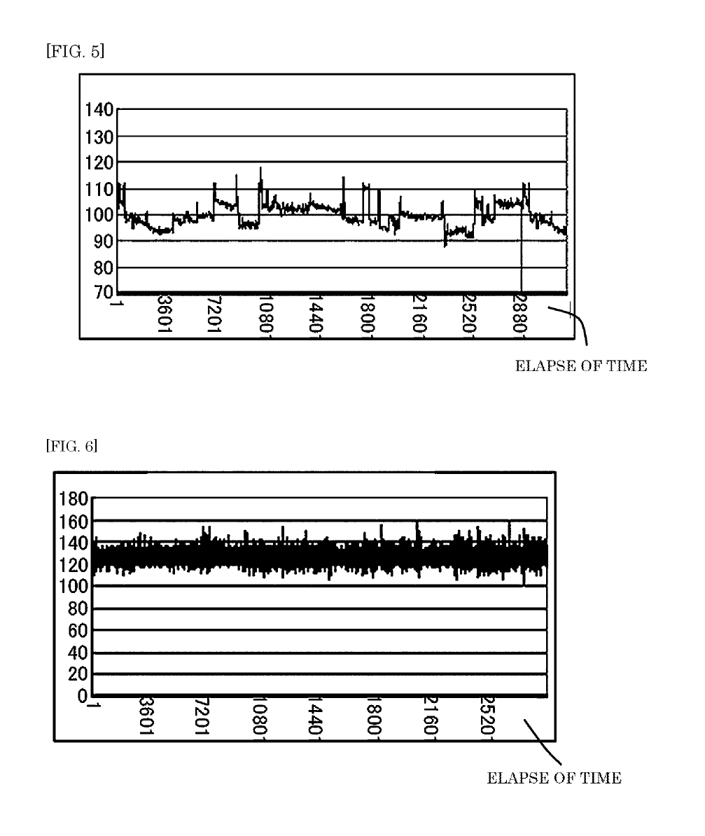

The second normalization unit 7 normalizes the data of the heartbeat intensity HI that has been calculated by the signal intensity calculation unit 5 so that the amplitude is included in a predetermined measurement range. Specifically, the second normalization unit 7 obtains the movement average value relative to the time intervals of 60 seconds as to the data relative to all the time intervals of the heartbeat intensity HI; further obtains an average value of the movement average value; multiplexes, by 100 times, a value obtained by dividing the movement average value by the average value to thereby perform normalization; and obtains the data of the second normalized heartbeat intensity HIN2 as shown in FIG. 5. The second normalization unit 7 performs such processing operation while shifting the data on a one by one second basis. Incidentally, the second normalized heartbeat intensity HIN2 is employed for determination of the awaking stage, the REM sleep stage, and the deep non-REM sleep stage. The second normalization unit 7 supplies the normalized data of the second normalized heartbeat intensity HIN2 to the sleep stage determination unit 10.

The third normalization unit 8 normalizes the data of the first normalized heartbeat intensity HIN1 that has been obtained by the first normalization unit 6 so that the amplitude is included in a predetermined range. Specifically, the third normalization unit 8 obtains a maximum value and a minimum value as to the data relative to all the time intervals of the first normalized heartbeat intensity HIN1; adjusts a difference between the maximum value and the minimum value so as to be 60% in width; and obtains data of the third normalized heartbeat intensity HIN3 as shown in FIG. 6. Incidentally, although the third normalized heartbeat intensity HIN3 is employed for determination of the awaking stage and the REM-sleep stage, data relative to time intervals of one hour at an initial stage of sleep and a signal of its large amplitude, which is generated at the time of clinical period, are excluded. The third normalization unit 8 supplies the normalized data of the third normalized heartbeat intensity HIN3 to the variance calculation unit 9.

The variance calculation unit 9 calculates the variances HIND1 and HIND3 that are indicative of variations of the data relative to a predetermined period of time as to the data of the first normalized heartbeat intensity HIN1 and the third normalized heartbeat intensity HIN3 that have been obtained by the first normalization unit 6 and the third normalization unit 8, respectively. Incidentally, in the embodiment, at a given time point, if an index indicative of variation of the data sampled within a given period of time leading up to that time point is referred to as a variance, a standard deviation of the data is employed as a variance. Specifically, the variance calculation unit 9, assuming that data of signal intensity is measured on one by one second basis, calculates the variance of the data relative to time intervals of 60 seconds, for example, among the data of a series of signal intensities. In this case, processing operations are repeatedly performed in such a manner as to calculate data relative to time intervals of 60 seconds dating back from a given time point, that is, the variance of 60 items of heartbeat data, and subsequently, calculate the variance for time intervals of 60 second dating back from the next one second after. As a result, the variance calculation unit 9 can obtain the time series data relative to time intervals of one second as to the variation (variance) of the signal intensity. For example, the variance calculation unit 9 obtains the data of the variance HIND1 as shown in FIG. 7 as to the first normalized heartbeat intensity HIN1 shown in FIG. 4, and obtains the data of the variance HIND3 as shown in FIG. 8 as to the third normalized heartbeat intensity HIN3 shown in FIG. 6. The variance calculation unit 9 supplies the thus obtained time series data to the sleep stage determination unit 10.

The sleep stage determination unit 10 determines a sleep stage of a user at the time of sleep, that is, any one of four stages consisting of the awaking stage, the REM-sleep stage, the shallow non-REM sleep stage, and the deep non-REM sleep stage, on the basis of the time series data of the second normalized heartbeat intensity HIN2 and the variances HIND1 and HINDS of the heartbeat intensity. Incidentally, when a body motion arises, a signal fluctuates greatly and the variance HIND of that signal intensity increases as well. Accordingly, in order to eliminate influence of such abnormal value, the sleep stage determination unit 10 performs abnormal value processing such as replacing the variance HIND of the signal intensity exceeding a predetermined value with the one meeting the predetermined value. In addition, the sleep stage determination unit 10 outputs information pertinent to the determined sleep stage, causes a display device, which is not shown, to display the output information or causes a printer to print the information, or causes a storage device to store the information as data. Incidentally, the processing operations by the sleep stage determination unit 10 will be later described in detail.

Such a sleep stage determination apparatus amplifies, by the signal amplification unit 2, the physiological signal that has been obtained by acquiring; detects the physiological signal by the physiological signal detection unit 1; and eliminates an unnecessary signal by the filter unit 3, by the band-pass filter or the like to thereby detect a heartbeat signal. In addition, in the sleep stage determination apparatus, the intensity HI of the heartbeat signal is calculated by the signal intensity calculation unit 5 while gain control is performed by the automatic gain control unit 4; as to the calculated intensity of the heartbeat signal, normalization is performed by the first normalization unit 6, the second normalization unit 7, and the third normalization unit 8; and on the basis of the variances HIND1 and HIND3 that have been calculated by the variance calculation unit 9 as to the obtained normalized heartbeat intensity HIN2 and the data of the normalized heartbeat intensities HIN1 and HIN3, determination of a sleep stage is performed by the sleep stage determination unit 10.

Such a sleep stage determination apparatus first performs determination of the awaking stage and subsequently performs determination of the REM-sleep stage and further performs determination of the deep non-REM sleep stage, by the sleep stage determination unit 10. The sleep stage determination apparatus performs a determination correction process, which will be described later, in determination of the deep non-REM sleep stage, and according to the result, corrects a result of determination of the REM sleep stage as well. In addition, the sleep stage determination apparatus, when determining time intervals of the awaking stage, the REM sleep stage, and the deep non-REM sleep stage, determines that the remaining time intervals obtained by subtracting the data relative to the time intervals that has been determined to be the awaking stage, the data relative to the time intervals that has been determined to be the REM sleep stage, and the data relative to the time intervals that has been determined to be the deep non-REM sleep stage from the data relative to all the sleep time intervals are the time intervals for the shallow non-REM sleep stage. The sleep stage determination apparatus performs determination of each stage by the sleep stage determination unit 10 as follows.

First, determination of the awaking stage will be described.

The sleep stage determination unit 10 determines the awaking stage on the basis of the second normalized heartbeat intensity HIN2 and the variance HIND3 of the third normalized heartbeat intensity HIN3. This advantage is that the threshold value of determination of the awaking stage can be uniformed. Specifically, the sleep stage determination unit 10 employs 8% of the variance HIND3 of the third normalized heartbeat intensity HIN3 as a common threshold value. That is, the time intervals at which the variance HIND3 is 8% or more of the threshold value are determined as candidates for the awaking stage.

More specifically, if the variance HIND3≥W1 (=8%) and the second normalized heartbeat intensity HIN2≥100−W2 (=4%), the sleep stage determination unit 10 determines the awaking stage when the detection time of that state is 10 seconds, the continuation time of that stage is 100 seconds, and the generation time period of that state is 700 seconds. On the other hand, if the variance HND3≥W1 (=8%) and the second normalized heartbeat intensity HIN2<100×W2, the sleep stage determination unit 10 determines the awaking stage when the detection time of that state is 10 seconds and the continuation time of that state is 100 seconds. The sleep stage determination unit 10, as is the case with the second normalized heartbeat intensity HIN2≥100×W2, takes a plurality of waveforms as one group and then determines that time intervals to be the awaking stage, or alternatively, if the second normalized heartbeat intensity HIN2<100−W2, determines the awaking stage on the basis of the individual single waveforms without using the generation time period. This processing operation will be described by way of specific signal example as follows.

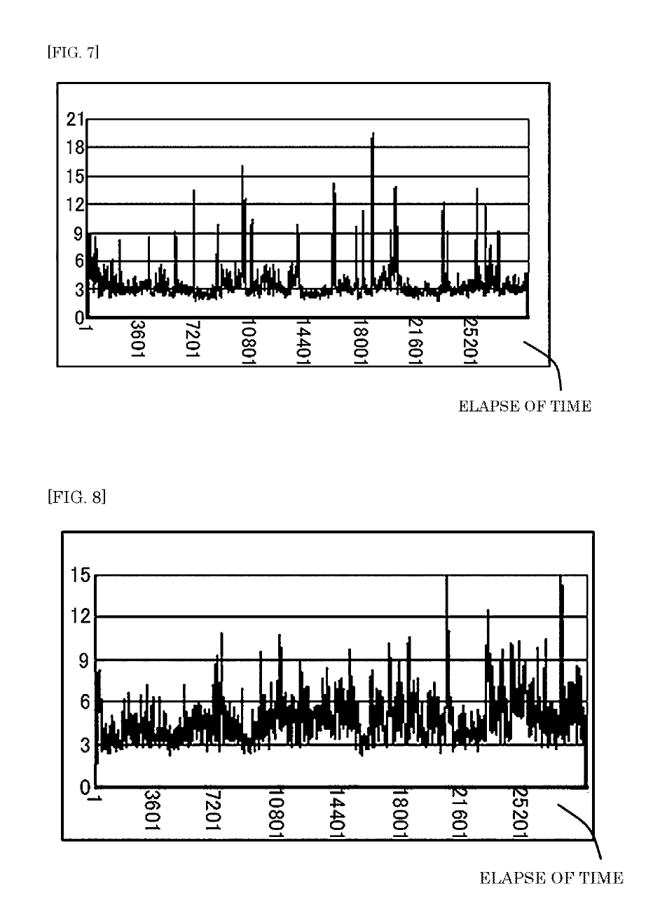

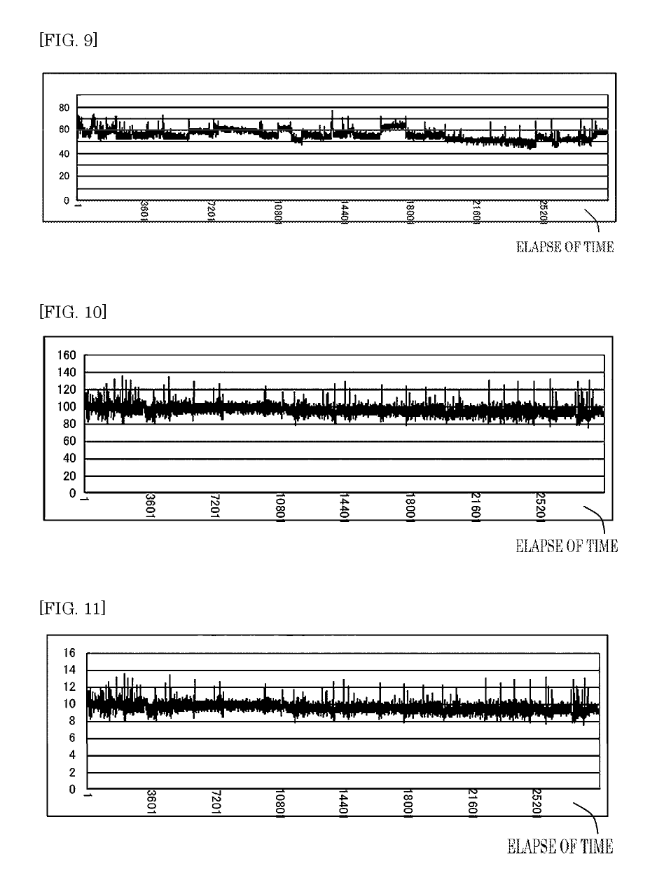

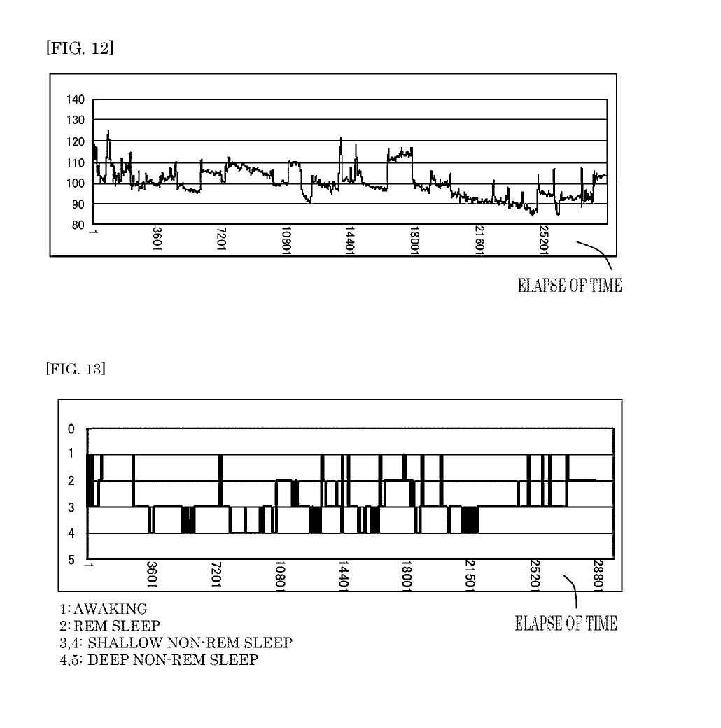

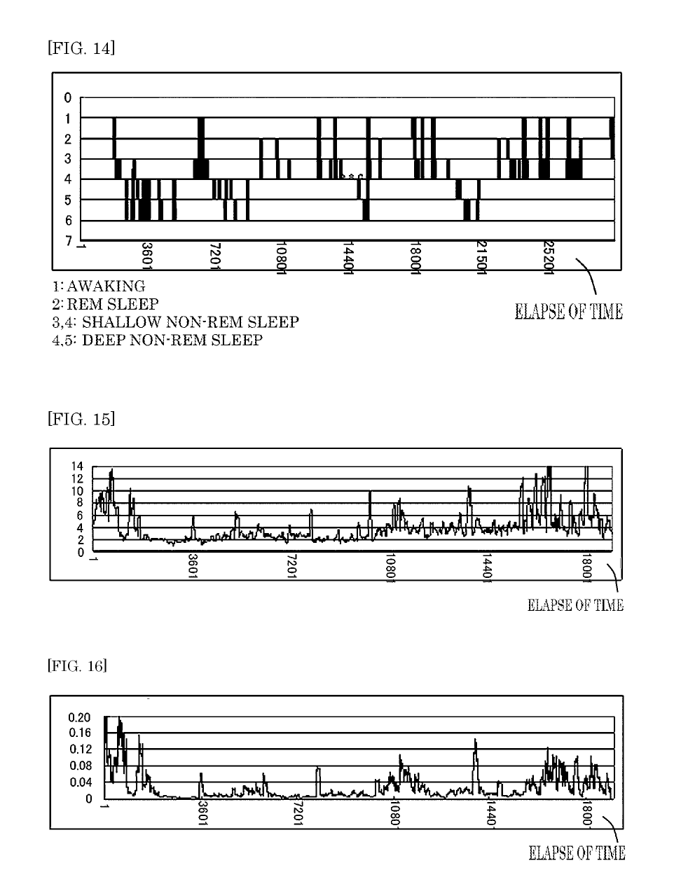

In consideration of the time series data of the heartbeat intensity HI as shown in FIG. 9, the third normalized heartbeat intensity HIN3 as shown in FIG. 10 is obtained, and the variance HIND3 is obtained as shown in FIG. 11. In addition, the second normalized heartbeat intensity HIN2 is obtained as shown in FIG. 12. If time intervals meeting the conditions described above are determined to be the awaking stage on the basis of these items of data, the result of determination as shown in FIG. 13 is obtained. Incidentally, the result of determination by the PSG is obtained as shown in FIG. 14, and the result close to the result of determination shown in FIG. 13 was obtained.

Next, determination of the REM sleep stage will be described.

Determination of the REM sleep stage is performed by utilizing the fact that an interrelationship between an autonomic nervous component and each of the peak intervals of a heartbeat signal is 80% or more and the variance of the heartbeat intensity and the sympathetic nerve component that has been obtained from the peak intervals of the heartbeat signal are associated with each other. Incidentally, the peak interval signal of the heartbeat signal is a signal obtained when the intervals of the waveform (R-wave) in the vicinity where the intensity of the heartbeat signal is peaked are employed as a variable, and in analysis of a heartbeat variation, this signal is frequently used as an R-R interval signal which is representative of the intervals of the adjacent peaks of the R-wave. In addition, a relationship between the peak interval signal and the autonomic nervous component is based on the fact that the power spectrum density that has been calculated by applying frequency analysis such as fast Fourier transform to the peak interval signal indicates an appearance which is different depending on the state of the autonomic nervous system. That is, the power spectrum density of the peak interval signal is characterized in that significant maximum values appear in a bandwidth of substantial 0.05 Hz to 0.15 Hz and in a bandwidth of substantial 0.2 Hz to 0.35 Hz; and however, assuming that the maximum value in the bandwidth of substantial 0.05 Hz to 0.15 Hz is referred to as an LF value and the maximum value in the bandwidth of substantial 0.2 Hz to 0.35 Hz is referred to as an HF value, these HF value and LF values are parameters which are indicative of the state of activities of the autonomic nerve, and when the LF value is large and the HF value is small, it indicates that the sympathetic nervous system is active and falls into a tense state, and when the LF value is small and the HF value is large, it indicates that the activities of the parasympathetic nervous system is active. Although the pulse rate decreases while in sleep, this is due to a decrease in the activities of the sympathetic nervous system that is active when it becomes tense and an increase in the activities of the parasympathetic nervous system that is active when it becomes relaxed. That is, it follows that the HF value and the LF value are significantly varied according to the state of depth of sleep. Specifically, the data of the LF value that corresponds to the data of the variance of the signal intensity as shown in FIG. 15 is obtained as shown in FIG. 16; these items of data are highly associated with each other; and both of the activities of the sympathetic nervous system and the variance of the heartbeat intensity in the vicinity of 11,000 seconds and 16,000 seconds become active. If a long term movement averaging process is applied to the time series data, the time intervals at which the activities of the sympathetic nervous system and the variance of the heartbeat intensity are active are obtained as the maximum value. The sleep stage determination unit 10 makes determination by utilizing the fact that the time intervals are equivalent to the REM sleep stage.

That is, the sleep stage determination unit 10 determines the REM sleep stage on the basis of the second normalized heartbeat intensity HIN2 and the variance HIND3 of the third normalized heartbeat intensity HIN3.

Specifically, the sleep stage determination unit 10 specifies and eliminates the time intervals of which value rapidly varies due to a change of a sleep state and a body motion (the time intervals at which the variance HIND3 of the third normalized heartbeat intensity HIN3 is changed by 7% or more) on the basis of the variance HIND3 of the third normalized heartbeat intensity HIN3. Specifically, the sleep stage determination unit 10 obtains a position of a maximum value of the variance HIND3 of the third normalized heartbeat intensity HIN3, and replaces the time intervals from 50 seconds ago to the maximum value to the value of 50 seconds ago. In addition, the sleep stage determination unit 10 replaces the time intervals from the position of the maximum value to 50 seconds later with the value of 50 seconds later. In addition, when the value after the replacement is smaller than the average value, the sleep stage determination unit 10 obtains an average value of the variance HIND3 of the third normalized heartbeat intensity HIN3 to thereby eliminate the time intervals of which value varies due to the state of the sleep state and the body motion. If the sleep stage determination unit 10 does not perform such processing operation, a number of maximum values, which will be described later, appear, making it difficult to specify the REM sleep stage.

Subsequently, the sleep stage determination unit 10 obtains a long term movement average value R0 (600 seconds) of the variance HIND3 of the third normalized heartbeat intensity HIN3, and obtains the maximum values. In addition, from among the thus obtained maximum values, the sleep stage determination unit 10 eliminates a maximum value which is generated due to the change of the sleep state, and determines the REM sleep stage. That is, the sleep stage determination unit 10 determines the REM sleep stage as to the time intervals at which the second normalized heartbeat intensity HIN2≥100−W2 (=4%) at a portion in the vicinity of the maximum value and at a portion of the maximum value. Incidentally, when the maximum value is 100%, the sleep stage determination unit 10 determines the REM sleep stage as to the time intervals at which the long term movement average value of the second normalized heartbeat intensity HIN2 is R1 (90%). In addition, as to the REM sleep stage, the time intervals of which average value is equal to or more than the long term movement average value of the variance HIND3 of the third normalized heartbeat intensity HIN3 are determined as the REM sleep stage. This processing operation will be described by way of specific signal example as follows.

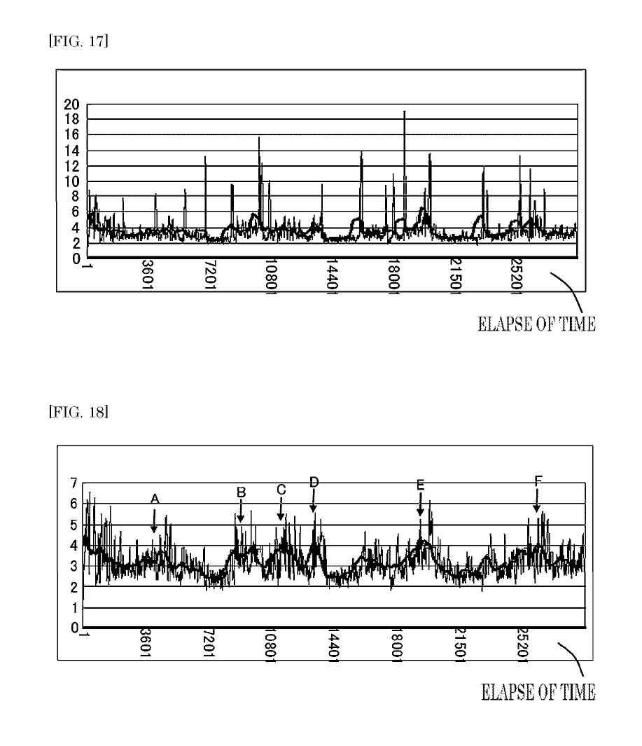

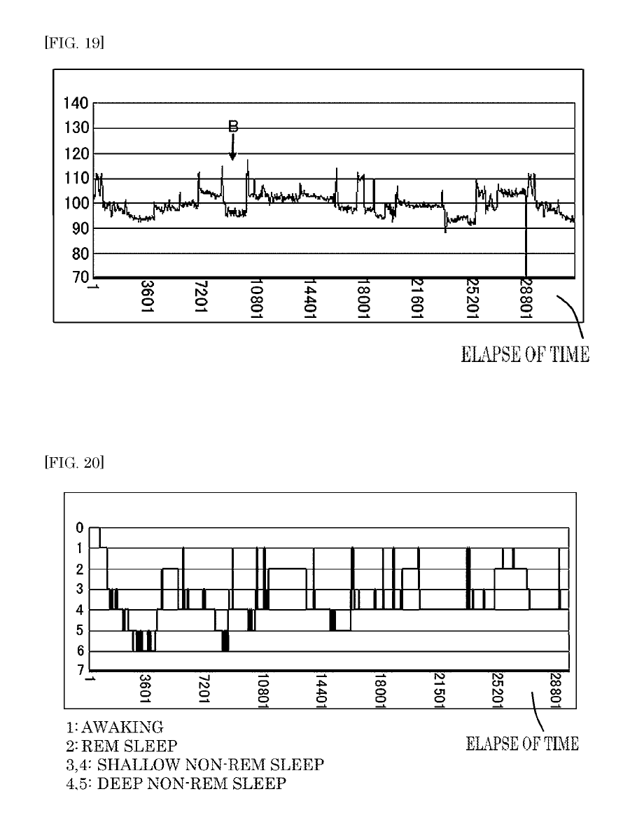

In consideration of the time series data of the variance HIND3 of the third normalized heartbeat intensity HIN3 as shown in FIG. 17, if no appropriate measure is taken, a number of maximum values appear, making it difficult to specify the REM sleep stage; and therefore, the sleep stage determination unit 10 eliminates the time intervals which rapidly varies due to the change of the sleep stage and the body motion, and obtains the data as shown in FIG. 18. In addition, the sleep stage determination unit 10, as shown in FIG. 18, obtains the long term movement average value R0 (600 seconds) of the variance HIND3, and obtains the maximum values. In this case, candidates A to F of which values each are equal to or larger than the average value 3.6 of all the long term movement average values R0 of 600 seconds are candidates for the maximum value. Among them, a candidate B is eliminated because the second normalized heartbeat intensity HIN2<100−W2, as shown in FIG. 19. Incidentally, the result of determination by adopting the PSG is obtained as shown in FIG. 20, and the result close to the result of determination shown in FIG. 18 was obtained.

Next, determination of the deep non-REM sleep stage will be described.

The sleep stage determination unit 10 determines the deep non-REM sleep stage on the basis of the variance HIND1 of the first normalized heartbeat intensity HIN1 and the second normalized heartbeat intensity HIN2.

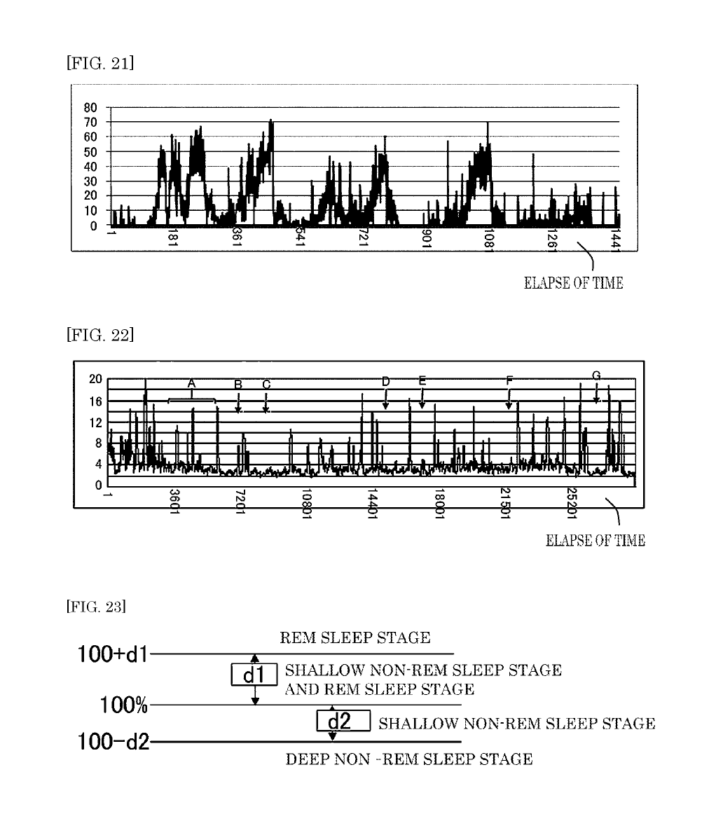

Specifically, the sleep stage determination unit 10 obtains a temporary time interval (maximum time interval) of the deep non-REM sleep stage as a primary determination process. First, the sleep stage determination unit 10 determines a threshold value of the deep non-REM sleep stage as to a predetermined rate of the average value AVHID of the data relative to all the time intervals of the variance HIND1 of the first normalized heartbeat intensity HIN1. Specifically, assuming that the average value AVHID of the data relative to all the time intervals of the variance HIND1 is 4.2, the sleep stage determination unit 10 determines 3.1 which is 75% of all, as a threshold value, and determines the time intervals of which value is equal to or less than the threshold value, as the non-REM sleep stage. However, even as to the time intervals of which value is equal to or larger than the threshold value, the sleep stage determination unit 10 determines 4.1%, which is greater by +1%, as a threshold value with respect to the average value AVHID of the variance HIND1 after noise has been eliminated. Also, in addition to such threshold value conditions, the sleep stage determination unit 10 determines the deep non-REM sleep stage as to the time intervals at which the continuation time of that state is within 40 seconds and 600 seconds having elapsed after that state has been established. That is, the sleep stage determination unit 10 determines the non-REM sleep stage as to the time intervals of 600 seconds after the threshold value conditions have been met. As shown in FIG. 21, this is determined in consideration of the fact that a rise time requires about 600 second in 6-wave component exerted by analysis of brain waves in which 20% or more is determined as the deep non-REM sleep stage. For example, in consideration of the time series data of the variance HIND1 as shown in FIG. 22, candidates A to G are those intended for the deep non-REM sleep stage.

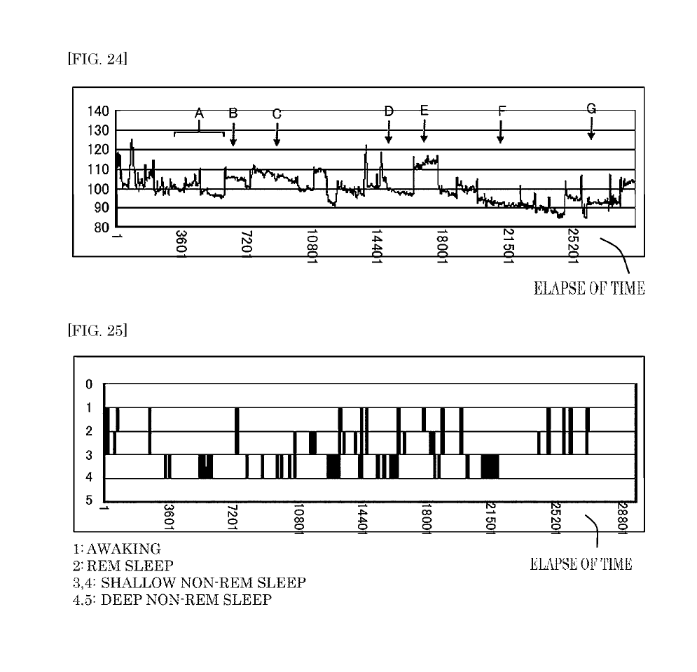

Next, as a second determination process, the sleep stage determination unit 10 performs a correction process of the result of the primary determination process on the basis of the second normalized heartbeat intensity HIN2. Specifically, as shown in FIG. 23, if d1=7% and d2=2%, the sleep stage determination unit 10 determines the shallow non-REM sleep stage as to the time intervals at which 0<MAVHINSE<2 if MAVHINSE obtained from the second normalized heartbeat intensity HIN2 (=MAVHIN (a starting edge of the deep non-REM sleep stage)−(MAVHIN+1 (a terminal edge of the deep non-REM sleep stage)<2%, and determines the REM sleep stage as to the time intervals at which MAVHINSE≤0. In addition, the sleep stage determination unit 10 determines the deep non-REM sleep stage if MAVHINSE≥2%. Therefore, assuming that the time series data of the second normalized heartbeat intensity HIN2, which corresponds to the time series data of the variance HIND1 shown in FIG. 22, is obtained as shown in FIG. 24, the sleep stage determination unit 10 determines, from among candidates A to G, the deep non-REM sleep stage as to candidates A, C, D, F that meet the second determination conditions.

The sleep stage determination unit 10 performs such processing operation to be thereby able to determine the time intervals of the awaking stage, the REM sleep stage, and the deep non-REM sleep stage. In addition, the sleep stage determination unit 10 determines the non-REM sleep stage as to the remaining time intervals. As an example, when a variety of stages were obtained by the technique according to the present invention, the result as shown in FIG. 25 was obtained. When comparison with the result of determination by adopting the PSG corresponding thereto was performed, it became as shown in Table 1 below, and the well coincident results were obtained.

TABLE 1

| | | REM | Shallow | Deep | |
|---|---|---|---|---|---|
| | Awaking | sleep | non-REM | non-REM | |
| | stage | stage | sleep | sleep | Total |
| PSG | 10.9% | 15.4% | 57.7% | 16.0% | 100% |
| The present | 9.6% | 15.9% | 55.7% | 18.8% | 100% |
| invention | | | | | |

As has been described hereinabove, the sleep stage determination apparatus shown as the embodiment of the present invention is characterized in that a variety of sleep stages are determined on the basis of the variance of the heartbeat intensity that has been obtained by performing an appropriate normalization process as to the heartbeat intensity; and therefore, it is possible to perform universal measurement free of an individual difference or an equipment difference, and it is possible to determine a sleep stage with its high accuracy in conformity with the international criterion for determining the depth of sleep as well.

Incidentally, the present invention is not limitative to the above described embodiment.

For example, as a method for detecting a heartbeat signal, the above described embodiment showed a method for extracting a heartbeat signal from a physiological signal which has been obtained by the nonrestrictive, physiological signal detection unit 1 having been provided under the user's body; and however, the present invention is applicable as long as there exists detection means by which a heartbeat signal or a signal equivalent to the heartbeat signal is continuously obtained. For example, according to the present invention, another physiological signal detection unit 1 is applicable as long as it serves as a heartbeat meter or a pulse meter of a wearable type on the user's wrist or upper arm or the like and is capable of continuously recording data.

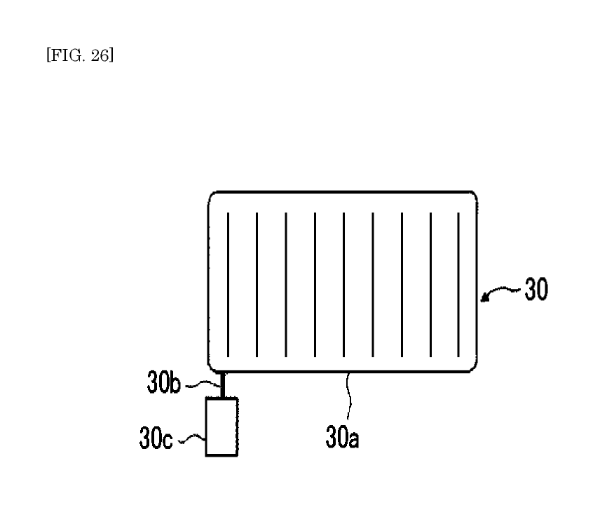

In addition, as the physiological signal detection unit 1, in place of employing the hollow tube described above, detection means of an air mat type as shown in FIG. 26 may be employed. That is, the physiological signal detection unit 30 shown in FIG. 26 is configured so that the air tube 30b is connected to one end of the air mat 30a containing air therein and further the fine differential pressure sensor 30c is connected to the air tube 30b. Incidentally, as the fine differential pressure sensor 30c, there can be employed the one similar to that described in the case of the physiological signal detection unit 1 employing the hollow tube.

Further, the above described embodiment employed a standard deviation as a variance indicative of variation of the heartbeat intensity; and however, according to the present invention, a statistical quantity such as a variance, a sum of deviation squares, or a predetermined range may be employed.

Needless to say, therefore, according to the present invention, appropriate alterations or modifications can occur without deviating from the spirit thereof.

### DESCRIPTION OF REFERENCE NUMERALS

1, 30 Physiological signal detection units
1a Pressure detection tube
1b, 30c Fine differential pressure sensors
2 Signal amplification unit
3 Filter unit
4 Automatic gain control unit
5 Signal intensity calculation unit
6 First normalization unit
7 Second normalization unit
8 Third normalization unit
9 Variance calculation unit
10 Sleep stage determination unit
21 Bed
22 Hard sheet
23 Cushion sheet
30a Air mat
30b Air tube

1. A sleep stage determination apparatus for determining a sleep stage of a user on a basis of a heartbeat signal which has been detected in a noninvasive and nonrestrictive manner at a time of sleep, the sleep stage determination apparatus comprising:
heartbeat signal detection means for detecting the heartbeat signal of the user in a noninvasive and nonrestrictive manner;
first normalization means for performing gain control relative to the heartbeat signal that has been detected by the heartbeat signal detection means to thereby uniformly control a peak value and then applying a first normalization process to an intensity of a heartbeat signal which has been calculated by employing a value of a gain at the time;
second normalization means for applying a second normalization process to the intensity of the heartbeat signal;
third normalization means for applying a third normalization process to the first normalized heartbeat intensity that has been obtained by the first normalization means;
variance calculation means for calculating a variance which is indicative of variation of data of a predetermined period of time as to data of the first normalized heartbeat intensity and the third normalized heartbeat intensity that have been obtained by the first normalization means and the third normalization means, respectively; and
sleep stage determination means for determining a sleep stage of the user on a basis of the second normalized heartbeat intensity that has been obtained by the second normalization means and a variance of the first normalized heartbeat intensity that has been calculated by the variance calculation means and a variance of the third normalized heartbeat intensity,
wherein the sleep stage determination means performs determination of an awaking stage on the basis of the second normalized heartbeat intensity that has been obtained by the second normalization means and the variance of the third normalization intensity that has been calculated by the variance calculation means,
performs determination of a REM sleep stage on the basis of the second normalized heartbeat intensity that has been obtained by the second normalization means and the variance of the third normalized heartbeat intensity that has been calculated by the variance calculation means,
performs determination of a deep non-REM sleep stage on the basis of the variance of the first normalized heartbeat intensity that has been calculated by the variance calculation means and the second normalized heartbeat intensity that has been obtained by the second normalization means, and
determines that a remaining time interval obtained by subtracting data relative to a time interval which has been determined to be a awaking stage, data relative to a time interval which has been determined to be a REM-sleep stage, and data relative to a time interval which has been determined to be a deep-non REM sleep stage, from data relative to all of sleep time intervals is a time interval of a shallow non-REM sleep stage.

2. The sleep stage determination apparatus according to claim 1, wherein the first normalization means obtains a movement average value of a predetermined period of time as to data of the intensity of the heartbeat signal and further obtains an average value of the movement average value and multiplexes, by 100 times, a value obtained by dividing the movement average value by the average value to thereby perform the first normalization process.

3. The sleep stage determination apparatus according to claim 1, wherein the second normalization means obtains a movement average value of a predetermined period of time as to data relative to all of time intervals of the intensity of the heartbeat signal and further obtains an average value of the movement average value and multiplexes, by 100 times, a value obtained by dividing the movement average value by the average value to thereby perform the second normalization process.

4. The sleep stage determination apparatus according to claim 1, wherein the third normalization means obtains a maximum value and a minimum value as to data relative to all of time intervals of the first normalized heartbeat intensity, and adjusts a difference between the maximum value and the minimum value so as to be a predetermined width to thereby perform the third normalization process.

5. A sleep stage determination method of determining a sleep stage of a user on the basis of a heartbeat signal which has been detected in a noninvasive and nonrestrictive manner at a time of sleep, the sleep stage determination method comprising:
a heartbeat signal detection step of detecting the heartbeat signal of the user in a noninvasive and nonrestrictive manner by predetermined heartbeat signal detection means;
a first normalization step of causing a processor, which performs signal processing, to perform gain control relative to the heartbeat signal that has been detected by the heartbeat signal detection step to thereby uniformly control a peak value, and then, apply a first normalization process to an intensity of a heartbeat signal which has been calculated by employing a value of a gain at the time;
a second normalization step of causing the processor to apply a second normalization process to the intensity of the heartbeat signal;
a third normalization step of causing the processor to apply a third normalization process to the first normalized heartbeat intensity that has been obtained in the first normalization step;
a variance calculation step of causing the processor to calculate a variance which is indicative of variation of data of a predetermined period of time as to the first normalized heartbeat intensity and the third heartbeat intensity that have been obtained in the first normalization step and the third normalization step, respectively;
a sleep stage determination step of causing the processor to determine a sleep stage of the user on the basis of the second normalized heartbeat intensity that has been obtained in the second normalization step and the variance of the first normalized heartbeat intensity and the variance of the third normalized heartbeat intensity that have been calculated in the variance calculation step,
wherein, in the sleep stage determination step, the processor performs determination of a sleep stage on the basis of the second normalized heartbeat intensity that has been obtained in the second normalization step and the variance of the third normalized heartbeat intensity that has been calculated in the variance calculation step,
performs determination of a REM sleep stage on the basis of the second normalized heartbeat intensity that has been obtained in the second normalization step and the variance of the third normalized heartbeat intensity that has been calculated in the variance calculation step, performs determination of a deep non-REM sleep stage on the basis of the variance of the first normalized heartbeat intensity that has been calculated in the variance calculation step and the second normalized heartbeat intensity that has been obtained in the second normalization step, and
determines that a remaining time interval obtained by subtracting data relative to a time interval which has been determined to be a awaking stage, data relative to a time interval which has been determined to be a REM sleep stage, and data relative to a time interval which has been determined to be a deep non-REM sleep stage, from data of all of time intervals of sleep is a time interval of a shallow non-REM sleep stage.
